(19) United States
(12) Patent Application Publication
(10) Pub. No.: US 2007/0224292 A1
(43) Pub. Date: Sep. 27, 2007

(54) Extended effect ergogenic drink

(76) Inventors: Daniel H. Brunner, Gurnee, IL (US); Frank Holas, Spring Grove, IL (US)

Correspondence Address: THE LAW OFFICE OF RANDALL T. ERICKSON, P.C., 1749 S. NAPERVILLE ROAD, SUITE 202, WHEATON, IL, 60187, US

(21) Appl. No.: 11/724,682

(22) Filed: Mar. 15, 2007

Related U.S. Application Data
(60) Provisional application No. 60/782,410, filed on Mar. 15, 2006.

(51) Int. Cl. A61K 31/555; A61K 31/522; A61K 31/455; A61K 31/192; A61K 31/375; A61K 33/14

(52) U.S. Cl. 424/678; 514/184; 514/263.34; 514/356; 514/474; 424/679; 424/680; 424/681; 514/568

(57) ABSTRACT

An extended energy drinkable beverage composition includes: caffeine, chromium in the form of chromium polynicotinate, niacin and the electrolytes calcium, potassium, magnesium, sodium, and the nutrient Vitamin C. The invention also relates to a method to use the composition, with the following effects: increased mental alertness, decreased fatigue, improved aerobic capacity, elevated mood, and increased overall circulation along with the associated benefits such as improved sensations of warmth in the extremities. The synergistic relationships of the caffeine, electrolytes, niacin, and chromium polynicotinate in this formulation result in an enhanced and prolonged ergogenic effect without the requirement of additional calories from carbohydrates.

This application claims the benefit of U.S. Provisional Application 60/782,410 filed Mar. 15, 2006.

### BACKGROUND OF THE INVENTION

1. Field of the Invention

This invention relates to an extended ergogenic formulation to provide nutrients and prolonged energy during mental and/or physical exertion without the requirement of additional calories from carbohydrates.

2. Description of Prior Work in the Field

Many energy and/or fluid replacement drink formulations provide carbohydrates and electrolytes such as sodium, potassium and chloride. Examples of such formulations include GATORADE and POWERADE. Also, most caffeinated formulations either have to reduce the caffeine levels or mask the bitter taste with high levels of artificial or natural sweeteners. Other formulations also require the use of carbohydrates to provide energy to, and sustain energy of, the consumer.

### SUMMARY OF THE INVENTION

This invention relates to an extended energy composition including: caffeine, chromium in the form of chromium polynicotinate, niacin and the electrolytes, calcium, potassium, magnesium, sodium, and the nutrient Vitamin C. The invention also relates to a method to use the composition, with the following effects: increased mental alertness, decreased fatigue, improved aerobic capacity, elevated mood, and increased overall circulation along with the associated benefits such as improved sensations of warmth in the extremities. The synergistic relationships of the caffeine, electrolytes, niacin, and chromium polynicotinate in this formulation result in an enhanced and prolonged ergogenic effect without the requirement of additional calories from carbohydrates.

The invention provides an ergogenic composition that can be delivered enterally thereby providing enhanced mental alertness, increased aerobic capacity, increased utilization of fat as an energy source, and to supplement with electrolytes and other key nutrients to enhance these effects or replace those that may have been lost during brief or sustained physical exertion. More specifically, the invention provides: an egogenic composition primarily consisting of caffeine, Potassium, Sodium, Magnesium, Calcium, Sodium Benzoate and/or Potassium Sorbate, Ascorbic and/or Citric Acid, Niacin, and Chromium Polynicotinate in an aqueous solution devoid of carbohydrates. As used herein, the term “carbohydrate” refers to monosaccharides, oligosaccharides, complex polysaccharides, or mixtures thereof.

An additional feature of this invention is the presence of niacin as a vasodilator and the biologically available form of chromium, Chromium Polynicotinate, to help maintain blood insulin levels. These ingredients are important to create an enhanced, ergogenic composition.

Particularly, the preferred embodiment of the invention provides an enhanced energy composition comprising, per 500 mL:

a) caffeine ranging from 0.05 g to 0.25 g;

b) niacin ranging from 2 mg to 200 mg;

c) Vitamin C and/or Citric Acid, total amount ranging from 10 mg to 2000 mg;

d) Chromium Polynicotinate or Chromium Picolinate ranging from 0.005 mg to 2 mg; and

e) Electrolytes totaling from 10 mg to 5000 mg, including Potassium Sorbate and/or Sodium Benzoate as a preservative, with ranges as follows:

I. Potassium Sorbate/Sodium Benzoate: 2 mg to 1000 mg

II. Magnesium Chloride: 2 mg to 1000 mg

III. Sodium Chloride: 2 mg to 1000 mg

IV. Calcium Chloride: 2 mg to 1000 mg

V. Potassium Chloride: 2 mg to 1000 mg.

As a further refinement of the preferred embodiment, the amount of caffeine is about 125 mg.

As a further refinement of the preferred embodiment, the elemental chromium is about 50 mcg.

As a further refinement of the preferred embodiment, the elemental calcium is about 25.2 mg.

As a further refinement of the preferred embodiment, the total elemental potassium is about 72.8 mg.

As a further refinement of the preferred embodiment, the elemental magnesium is about 20.4 mg.

As a further refinement of the preferred embodiment, the total elemental sodium is about 47.9 mg.

As a further refinement of the preferred embodiment, the citric acid is about 791 mg.

As a further refinement of the preferred embodiment, the niacin is about 9.9 mg.

As a further refinement of the preferred embodiment, the potassium sorbate is about 179 mg.

While many known energy and/or fluid replacement drink formulations provide carbohydrates and electrolytes such as sodium, potassium and chloride, none combine caffeine, niacin and chromium along with the electrolytes calcium, magnesium, sodium, potassium and chloride.

The energy drink formulation of the preferred embodiment of the invention utilizes a balance of electrolytes to counter the bitter taste of caffeine while still maintaining levels known to enhance athletic and mental performance without the use of sweeteners. This formulation design not only enhances but can prolong the desirable effects of caffeine while mitigating the less desirable ones as well.

While other known formulations use carbohydrates to provide energy to, and sustain energy of, the consumer, the formulation of the preferred embodiment of the invention utilizes caffeine in combination with niacin, chromium, and previously mentioned electrolytes to enhance and sustain the ergogenic effect.

Numerous other advantages and features of the present invention will be become readily apparent from the following detailed description of the invention and the embodiments thereof, from the claims and from the accompanying drawings.

### DETAILED DESCRIPTION OF THE INVENTION

While this invention is susceptible of embodiment in many different forms, there are shown in the drawings, and will be described herein in detail, specific embodiments thereof with the understanding that the present disclosure is to be considered as an exemplification of the principles of the invention and is not intended to limit the invention to the specific embodiments illustrated.

The invention provides an ergogenic composition that can be delivered enterally thereby providing enhanced mental alertness, increased aerobic capacity, increased utilization of fat as an energy source, and to supplement with electrolytes and other key nutrients to enhance these effects or replace those that may have been lost during brief or sustained physical exertion.

More specifically, the invention provides: an egogenic composition primarily consisting of Caffeine, Potassium, Sodium, Magnesium, Calcium, Potassium Sorbate, Ascorbic Acid, Niacin, and Chromium Polynicotinate. This invention does not require the use of carbohydrates to generate the immediate or prolonged ergogenic effects, but rather relies on the synergistic relationship of the specific components. This does not preclude the use of flavorings, colorings or sweeteners to enhance visual and taste appeal, but rather does not rely upon them, adding to the uniqueness of this invention. The use of non-carbohydrate sweeteners is the preferred form if choosing to flavor the formulation and, additionally, as a nutritive supplement, the use of Stevia Powder and/or extract as a synergistic component of the formula is recommended if formulating a flavored version of this invention.

In particular, the individual components are discussed in detail as follows:

Caffeine

The composition includes caffeine. Caffeine ranges from between about 50 to 200 mg, but preferably is 125 mg per 500 mL.

From: http://www.sleepfoundation.org/caffeine.cfm

Caffeine is a substance that is extracted from plants or synthetically produced. It is found in coffee, tea, cola (the three most popular drinks in the world) and some other sodas, as well as chocolate and certain medicines. It has been consumed in coffee, tea and chocolate for much of human history, but it wasn't officially discovered until 1819.

The pharmacological/physiological effects of caffeine include:

Improved Visual Monitoring:

Mitchell, Ross and Hurst showed caffeine to prevent attention lapses in a visual monitoring test which simulated night driving. The effect persisted for the two to three hour experiment (Stephenson, 1977).

STEPHENSON. P. E. Physiologic and Psychotropic Effects of Caffeine on Man. J. Am. Dietetic Assoc. 71, 240, Sepi, 1977.

Half Life Information:

Caffeine has a physiological half-life of three and a half hours (Parsons and Neims, 1978) to six hours (Aranda et al., 1979) and its physiological effects are observed in less than one hour. Women taking oral contraceptives can see an increase in half-life of up to 12 hours.

(Parsons an PARSONS. W. O. and NEIMS. A. H.: Effect of Smoking op Caffeine Clearance. J. Clan. Pharm. Their. 24, 1, 40-45, 1978.d Neims, 1978).

ARANDA. J. V. at al.: Pharmacokinetic Profile of Caffeine in the Premature Newborn Infant with Apnea. J. Pediatr. 94, 4, 663-666, 1979.

Blood Flow (Vaso-Reactive):

Although caffeine dilates blood vessels by a direct action, its central effect is one of constriction. At higher doses, the dilating effect is apparent (Peach, 1972; Poisner, 1973).

PEACH, M. J.: Stimulation of Release of Adrenal Catecholarnine by Adenosine CMP and Theophylline in the Absence of Extracellular Calcium. Proc. Nat. Aced. Sci., USA, 69,834-836, 1972.

POISNER, A. M.: Direct Stimulant Effect of Ammophylline on Catechola. mine Release from the Adrenal Medulla. Bioch. Pharmacol. 22, 469-476, 1973.

Caffeine's purported efficacy in hypertensive headaches may be due to a decrease in blood flow as a result of the increased cerebral resistance (Ritchie et al., 1975).

RITCHIE. M. J. GOODMAN. L. and GILMAN, A.: The Pharmacological Basis of Therapeutics. 5th Ed. MacMillan, N.Y., 1975.

Caffeine “Let Down”: After two hours, Klein reported that males (but not females) showed a lower CNS (Central Nervous System) stimulation compared to placebo. The post stimulation “let down” with caffeine results in fatigue and lethargy

KLEIN, R. H. and SALZMAN, L. F.: Paradoxical Effects of Caffeine Percept. and Motor Skills 40, 126. 1975.

From: http://www.priory.com/pharmol/caffeine.htm

Caffeine has been shown to increase circulating free fatty acids and glycerol during exercise in normal subjects, with associated improvements in exercise duration (Costill D L, Dalsky G P, Fink W J, 1978; Berglund B, Hemmingsson P., 1982) maximum oxygen consumption (Toner M. M et al, 1982) resting metabolic rate (LeBlanc J et al, 1985) or perceived exertion (Casal D C, Leon A S 1985).

Toner M. M., Kirkendall D. T., Dello D. J., Chase J. M., Clearly P. A., Fox E. L. Metabolic and cardiovascular responses to exercise with caffeine. Ergonomics 1982; 25:1175-1183.

Costill D L, Dalsky G P, Fink W J. Effect of caffeine ingestion on metabolism and exercise performance. Med Sci Sports 1978; 10:155-158.

Berglund B, Hemmingsson P. Effects of caffeine ingestion on exercise performance at low and high altitudes in cross country skiers. Int J Sports Med 1982; 3:234-236.

LeBlanc J, Jobhin M, Cote J, Samson P, Labrie A. Enhanced metabolic response to caffeine in exercise trained human subjects. J Appl Physiol 1985; 59:832-837.

Casal D C, Leon A S. Failure of caffeine to affect substrate utilization during prolonged running. Med Sci Sports Exerc 1985; 17:174-179.

Caffeine is rapidly absorbed through the stomach and small intestine into the bloodstream, where it takes about 15-45 minutes to reach peak levels. The level of caffeine in the blood reaching the brain determines the potency of its effects on the body. Caffeine will continue to have an effect on the body as long as it remains in the blood.

Caffeine and Niacin Relationship:

Niacin, through its use as a vasodilator (described in the Niacin section below) can improve blood flow to the brain thereby improving the potency of the caffeine. Further, the differential half-lives of Niacin (approximately 1 hour for immediate release Niacin) and Caffeine (3.5 to 6 hours) result in the removal of Niacin prior to Caffeine. The net effect is a reduced clearance of Caffeine due to restricted blood flow. Additionally, Niacin's effect on liver function may further augment this process (described in the Niacin section below). In summary, this synergistic relationship results in: a) improved potency of the caffeine, b) prolonged effects including sustained energy, and c) decreased clearance of caffeine to avoid the caffeine “let down” effect.

Sports Related Effects:

From: http://www.rice.edu/˜jenky/sports/caffeine.html

Caffeine causes the body to burn more fat and fewer carbohydrates. Glycogen is the principle fuel for muscles, but fat is the most abundant resource that the body uses for energy. Caffeine enters the body and forces the working muscles to utilize as much fat as possible. This delays the immediate depletion of glycogen. Studies show that in the first fifteen minutes of exercise caffeine has the potential to reduce the loss of glycogen by fifty percent. When this happens, the saved glycogen can be used for the remainder of the workout where normally it would be entirely depleted.

Niacin

The composition also includes Niacin. Niacin ranges from between about 2 to 200 mg, but preferably is 9.9 mg per 500 mL.

From: http://www.gettingwell.com/drug_info/nmdrugprofiles/nutsupdrugs/nia_{—}0184.shtml

Niacin is a member of the B-vitamin family. It is sometimes referred to as vitamin B₃. Nicotinic acid was first discovered as an oxidation product of nicotine and thus, the origin of its name. In fact, much of the confusion caused by the use of the term niacin for both nicotinic acid and nicotinamide, as well as for nicotinic acid alone, was created by the attempt to dissociate nicotinic acid from its nicotine origins. Niacin, via its metabolites, is involved in a wide range of biological processes, including the production of energy, the synthesis of fatty acids, cholesterol and steroids, signal transduction, the regulation of gene expression and the maintenance of genomic integrity.

The time to reach peak serum concentrations of the immediate-release or crystalline form of nicotinic acid is approximately 45 minutes following ingestion. The time to reach peak serum concentrations of the extended-release form of nicotinic acid is from 4-5 hours following ingestion. Administration of nicotinic acid with food maximizes its availability. Nicotinic acid-induced flushing, which is due to vasodilation, occurs within 20 minutes following ingestion of immediate-release nicotinic acid and may last for up to one hour.

Chromium

The composition also includes Chromium. Chromium ranges from between about 0.02 to 2 mg, but preferably is 0.05 mg (50 mcg) of elemental chromium (equivalent to 400 mcg chromium polynicotinate) per 500 mL. Mcg denotes micrograms. This formulation does not preclude the use of Chromium Picolinate, but the preferred form is Chromium Polynicotinate.

A biologically active form of chromium participates in glucose metabolism by enhancing the effects of insulin. Insulin is secreted by specialized cells in the pancreas in response to increased blood glucose levels, for example, after a meal. Insulin binds to insulin receptors on the surface of cells, activating those receptors and stimulating glucose uptake by cells. Through its interaction with insulin receptors, insulin provides cells with glucose for energy and prevents blood glucose levels from becoming elevated. In addition to its effects on carbohydrate (glucose) metabolism, insulin also influences the metabolism of fat and protein.

Chromium has been shown to improve insulin sensitivity at 200 to 400 micrograms (mcg) of chromium three times per day in the polynicotinate or picolinate form (Wilson, B E 1995)

Wilson, B E, et al. Effects of chromium supplementation on fasting insulin levels and lipid parameters in healthy, non-obese young subjects. Diabetes Res Clin Pract (1995) 28(3):179-184.

The preferred chromium in this composition is delivered via niacin bound chromium polynicotinate. Niacin bound chromium polynicotinate has been shown to lower blood lipid levels. U.S. Pat. Nos. 4,923,855 and 4,954,492 are hereby incorporated by reference.

Chromium is considered to be a part of the yet to be completely defined “glucose tolerance factor” (GTF), which is a water soluble constituent of liver, blood plasma, brewer's yeast and some other biological extracts and cells. It is suggested that GTF was a chromium-containing complex, along with nicotinic acid, glycine, glutamic acid, cysteine and glutathione.

Impaired glucose tolerance can be improved or normalized by chromium supplementation, or can be maintained in spite of a reduced insulin output. Chromium is an essential nutrient, not a drug. Chromium will not improve normal glucose tolerance.

The maintenance of adequate chromium stores is critical to insulin's effects on the metabolism of fats, carbohydrates and proteins, Due to excessive chromium turnover and often a marginal chromium intake, athletes may have a larger need for chromium.

Supplementation with chromium to restore and maintain sufficient chromium stores may help to promote optimal insulin efficiently—a necessity to high level athletic performance.

This organically bound complex of nicotinic acid and chromium is in fact suggested to be the “biologically” active form of chromium in the body. Recent advances in chromium nutrition research strengthen the association of insufficient dietary chromium as one of the risk factors to maturity onset, diabetes and cardiovascular diseases, once again stressing the role of chromium in maintenance of optimal health.

Chromium, as a trace mineral, plays a vital role in fighting stress and maintaining the immune system.

Chromium and Niacin References:
1. Lefavi R. G. Intl J of Sports Nutrition 2(2): 111-122, (1992)
2. Anderson, R. A. et al—Diabetes, 46(11): 1786-91 (1997)
3. Ofeen Bacher, E. G. et al. Diabetes, 29:919 (1980)
4. Elliot R B; Chase H P. Diabetologia, 34(5):362-5 (1991)
5. Mertz, Physiol. Rev 49, 103-238 (1969)
6. A. Ladenberg, Ann Chem. 301, 152 (1898)
7. S. M Mc Elvain, Org. Syn. Co11. Vol I, 385(1941)
8. C. F. Woodward et. al., Ind. Eng. Chem 36, 540 544(1944)
9. Capuzzi D M, Guyton J R, Morgan J M, et al. Am J Cardiol 82(12A): 74U-81U(1998)
10. Colletti R B, Neufeld E J, Rofff N K, et al. Pediatrics 92:78-82 (1993)
11. Elam M B, Hunninghake, D B, Davis K B, et al. JAMA. 284:1263-1270 (2000)
12. Guyton J R. Am J cardiol 82:18 U-23U.(1998)
13. Mirsky, N. Harefuah, 132(2): 133-6 (1997)
14. J. A. Cooper, B. F. Anderson et. al., Inorg. Chim. Acta 91, 1 (1984)
15. Schwartz K and Mertz W Arch. Biochim. Biophy 85 292 (1959)
16. Mertz W, Toepfer E W et. al Federation Proc. 3 2275 2280 (1975)
17. Chang et. al Inorg. Chem 22 1739 44. (1983

Chromium in the form of chromium polynicotinate, can help maintain steady blood glucose levels through improved insulin sensitivity and the reduction of “insulin spikes”. All aspects of fat metabolism are greatly enhanced by an absence of excessive insulin (as in an insulin spike). In the absence of excess insulin, the enzyme hormone sensitive lipase in the fat cells becomes activated, causing hydrolysis of the stored triglycerides, releasing large quantities of fatty acids and glycerol into the blood stream. The free fatty acids then become the main source of fuel to be burned as energy. So in the absence of insulin the fat stores are burned as well as a free fatty acids being released into the blood stream for use as an energy source. In regards to muscle anabolism, the importance of free fatty acids are apparent. Low serum FFA (free fatty acid) levels increase muscle glycogenolysis (muscle breakdown), especially crucial during exercise.

The use of chromium in this invention, in regards to the FFA levels for use as an energy source and reduction in glycogenolysis, is additive to the FFA release experienced during caffeine consumption as stated earlier.

Vitamin C (Ascorbic Acid) and/or Citric Acid

The composition may include Ascorbic Acid. Ascorbic Acid/Citric Acid ranges from between about 10 to 2000 mg, but preferably is 831 mg per 500 mL. The use of Citric Acid, another antioxidant, may also be used alone or in combination with Ascorbic Acid to total the amounts listed above and both serve as acidulants in the formulation.

Vitamin C and Chromium: Chromium uptake is enhanced in animals when given at the same time as vitamin C (Stoecker, 1999). In a study of three women, administration of 100 mg of vitamin C together with 1 mg of chromium resulted in higher plasma levels of chromium than 1 mg of chromium without vitamin C (National Academy Press, 2001).

Stoecker B J. Chromium. In: Shils M, Olson J A, Shike M, Ross A C, eds. Nutrition in Health and Disease. 9th ed. Baltimore: Williams & Wilkins; 1999:277-282.

Food and Nutrition Board, Institute of Medicine. Chromium. Dietary reference intakes for vitamin A, vitamin K, boron, chromium, copper, iodine, iron, manganese, molybdenum, nickel, silicon, vanadium, and zinc. Washington, D.C.: National Academy Press; 2001:197-223. (National Academy Press)

Vitamin C is required for the synthesis of collagen, an important structural component of blood vessels, tendons, ligaments, and bone. Vitamin C also plays an important role in the synthesis of the neurotransmitter, norepinephrine.

Neurotransmitters are critical to brain function and are known to affect mood. In addition, vitamin C is required for the synthesis of carnitine, a small molecule that is essential for the transport of fat to cellular organelles called mitochondria, for conversion to energy (Carr A C, Frei B, 1999) Recent research also suggests that vitamin C is involved in the metabolism of cholesterol to bile acids, which may have implications for blood cholesterol levels and the incidence of gallstones (Simon J A, Hudes E S, 2000)

Carr A C, Frei B. Toward a new recommended dietary allowance for vitamin C based on antioxidant and health effects in humans. Am J Clin Nutr. 1999; 69(6):1086-1107. (PubMed)

Simon J A, Hudes E S. Serum ascorbic acid and gallbladder disease prevalence among US adults: the Third National Health and Nutrition Examination Survey (NHANES III). Arch Intern Med. 2000; 160(7):931-936. (PubMed)

In summary, the use of Vitamin C in this invention supports brain neurotransmitter synthesis and the synthesis of carnitine essential for the use of FFA for conversion to energy, enhances the uptake of chromium and serves to quench free radicals (oxygen reactive molecules) generated during exercise and the accompanying respiration.

Electrolytes

Calcium

Additionally, the composition includes the electrolyte calcium. Calcium ranges from between about 2 mg to 1000 mg, but preferably is 25.2 mg elemental calcium per 500 mL.

From: http://lpi.oregonstate.edu/infocenter/minerals/calcium/calciumrefs.html#ref3

On average, one 8-ounce cup of coffee (approx. 100 mg caffeine) decreases calcium retention by 2-3 mg. (Weaver, 1999)

Weaver C M, Heaney R P. Calcium. In: Shils M, Olson J A, Shike M, Ross A C, eds. Nutrition in Health and Disease. 9th ed. Baltimore: Williams &Wilkins; 1999:141-155.

Calcium plays a role in mediating the constriction and relaxation of blood vessels (vasoconstriction and vasodilation), nerve impulse transmission, muscle contraction, and the secretion of hormones, such as insulin (National Academy Press, 1997)

Food and Nutrition Board, Institute of Medicine. Calcium. Dietary Reference Intakes: Calcium, Phosphorus, Magnesium, Vitamin D, and Fluoride. Washington, D.C.: National Academy Press; 1997:71-145. (National Academy Press)

In summary, the electrolyte Calcium plays a valuable role in this invention. It supports the vaso-reactive properties of caffeine and niacin, it mediates muscle required during vigorous activities, it can counter some of the decreased calcium retention due to caffeine as well as mediate the secretion of hormones such as insulin and thereby support the role of Chromium.

Potassium

Another electrolyte in the composition is potassium. Potassium ranges from between about 2 mg to 1000 mg, but preferably is 72.8 mg elemental potassium per 500 mL.

Potassium is the principal positively charged ion (cation) in the fluid inside of cells, while sodium is the principal cation in the fluid outside of cells. Potassium concentrations are about 30 times higher inside than outside cells, while sodium concentrations are more than 10 times lower inside than outside cells. The concentration differences between potassium and sodium across cell membranes create an electrochemical gradient known as the membrane potential. A cell's membrane potential is maintained by ion pumps in the cell membrane, especially the sodium, potassium-ATPase pumps. These pumps use ATP (energy) to pump sodium out of the cell in exchange for potassium. Their activity has been estimated to account for 20%-40% of the resting energy expenditure in a typical adult. The large proportion of energy dedicated to maintaining sodium/potassium concentration gradients emphasizes the importance of this function in sustaining life. Tight control of cell membrane potential is critical for nerve impulse transmission, muscle contraction, and heart function (Sheng, H-W, 2000; Brody T, 1999)

Sheng H-W. Sodium, chloride and potassium. In: Stipanuk M, ed. Biochemical and Physiological Aspects of Human Nutrition. Philadelphia: W.B. Saunders Company; 2000:686-710.

Brody T. Nutritional Biochemistry. 2nd ed. San Diego: Academic Press; 1999.

Potassium Sorbate and Sodium Benzoate:

Potassium Sorbate and Sodium Benzoate are included in this composition as a preservative. Total preservative levies range from between about 2 mg to 1000 mg, but preferably is 257.9 mg per 500 mL.

Potassium sorbate is the potassium salt of sorbic acid and is much more soluble in water than the acid form. Sodium Benzoate is the sodium salt of Benzoic acid. When dissolved in water, potassium sorbate ionizes to form sorbic acid (which is effective against yeasts, molds, and select bacteria) and potassium. When dissolved in water, Sodium Benzoate ionizes to form benzoic acid and sodium. As such, these constituents are an effective preservative combination as well as another source of sodium and potassium upon dissociation.

Stevia/Sucralose:

If a flavored version is desired, Stevia may enhance moods and increase energy levels and mental alertness thereby acting as an additional synergistic component of this invention. It has also been shown to stop the growth of bacteria in the mouth is responsible for the production of acids that are responsible for gingivitis and cavities.

An exemplary level of stevia is within a range of about 20 to 80 mg, preferably about 65.7 mg.

If flavored, sucralose is the desired means of providing sweetness in this formulation as it doesn't provide additional calories or carbohydrates.

An exemplary level of sucralose is within a range of about 5 to 40 mg, preferably 20 mg.

Katayama O., Sumida, T, Hayashi, H. and Mitsuhashi H. 1976. The practical application of Stevia and research and development data (English translation). I.S.U. Company, Japan. 747 pp.

Das, S., Das, A. K., Murphy, R. A., Punwani, I. C., Nasution, M. D., and Kinghorn, A. D. 1992. Evaluation of the cariogenic potential of the intense natural sweeteners stevioside and rebaudioside A. Caries Res. 26: 363-366.

Magnesium:

Still another electrolyte included in this composition is magnesium. Magnesium ranges from between about 2 mg to 1000 mg, but preferably is 20.4 mg elemental Magnesium per 500 mL

General Role:

Magnesium is involved in more than 300 essential metabolic reactions, some of which are discussed below (Shils M E, 1999; Spencer H, Norris C, Williams D, 1994)

Energy Production:

The metabolism of carbohydrates and fats to produce energy requires numerous magnesium-dependent chemical reactions. Magnesium is required by the adenosine triphosphate (ATP) synthesizing protein in mitochondria. ATP, the molecule that provides energy for almost all metabolic processes, exists primarily as a complex with magnesium (MgATP).

Shils M E. Magnesium. In: Shils M, Olson J A, Shike M, Ross A C, eds. Nutrition in Health and Disease. 9th ed. Baltimore: Williams & Wilkins; 1999:169-192.

Spencer H, Norris C, Williams D. Inhibitory effects of zinc on magnesium balance and magnesium absorption in man. J Am Coll Nutr. 1994; 13(5):479-484. (PubMed)

Sodium and Chloride:

Still another electrolyte included in this composition is Sodium. Sodium ranges from between about 2 mg to 1000 mg, but preferably is 47.9 mg elemental Sodium per 500 mL

As a byproduct of salt dissociation, the counter ion chloride may be included in this composition, but is not considered essential for the invention. Sodium (Na⁺) and chloride (Cl⁻) are the principal ions in the fluid outside of cells (extracellular fluid), which includes blood plasma. As such, they play critical roles in a number of life-sustaining processes (Harper M E, Willis J S, Patrick J 1997)

Harper M E, Willis J S, Patrick J. Sodium and chloride in nutrition. In: O'Dell B L, Sunde R A, eds. Handbook of nutritionally essential minerals. New York: Marcel Dekker; 1997:93-116.

As previously discussed in the potassium section, the concentration differences between potassium and sodium across cell membranes create an electrochemical gradient known as the membrane potential. A cell's membrane potential is maintained by ion pumps in the cell membrane, especially the sodium, potassium-ATPase pumps. These pumps use ATP (energy) to pump sodium out of the cell in exchange for potassium. Their activity has been estimated to account for 20%-40% of the resting energy expenditure in a typical adult.

Hyponatremia is defined as a serum sodium concentration of less than 136 mmol/liter, and can be caused by an excessive loss of sodium. Hyponatremia has recently been recognized as a potential problem in individuals competing in very long endurance exercise events, such as marathons, ultramarathons, and Ironman triathlons. In 1997, 25 out of 650 participants in an Ironman triathlon (almost 4%) received medical attention for hyponatremia (Speedy D B, Rogers I R, Noakes T D, 2000) Symptoms of hyponatremia include headache, nausea, vomiting, muscle cramps, fatigue, disorientation, and fainting. Speedy D B, Rogers I R, Noakes T D, et al. Diagnosis and prevention of hyponatremia at an ultradistance triathlon. Clin J Sport Med. 2000; 10(1):52-58. (PubMed)

Electrolyte Summary:

In general, the supportive and synergistic role of these electrolytes work in tandem with the other components of this formulation to maximize muscle contraction, enhance recovery, support carbohydrate and fat metabolism, avoid mineral depletion, support nerve impulse generation, and mediate energy at the cellular level.

Although the Chloride formulation of the electrolyte salts is the preferred form for delivery to this invention, other counter ions such as citrate or sulfate may also be utilized.

Method(s) of Preparation:

The drinkable beverage composition may be prepared by mixing together all of the ingredients in dry form then dissolved in water or individually dissolved in water. The solution is agitated with a mechanical mixer until all of the ingredients have gone into solution. It may be necessary for the solution to be heated to a temperature between about 25.degree. C. and about 35 degree. C. to get all the ingredients dissolved. If undissolved particulate remains, the solution or concentrate may be filtered through an inert matrix of sufficient mesh or pore size to clarify and/or remove existing microbials.

The beverage may be prepared as a concentrated liquid and dissolved and clarified per above as required. The concentrate may then be adjusted to the final formulation with water and agitated to facilitate homogeneity.

After the mixture has been sufficiently dissolved and clarified if necessary, the beverage may be bottled, capped and labeled. To improve shelf life, this formulation may be heated and hot filled.

### EXAMPLE

Table 1 shows how to make 4 LT of a 10 Fold Orange Flavored Concentrate Composition within the scope of this invention. The diluted form of this composition is best administered prior to activities requiring heightened mental acuity or intense physical activity.

TABLE 1

| Ingredient | Weight | |
|---|---|---|
| CAFFEINE (ANHYDROUS) | 10.0 | GMS |
| POTASSIUM CHLORIDE | 8.0 | GMS |
| SODIUM CHLORIDE | 7.2 | GMS |
| CALCIUM CHLORIDE | 5.6 | GMS |
| MAGNESIUM CHLORIDE | 6.4 | GMS |
| POTASSIUM SORBATE | 14.3 | GMS |
| SODIUM BENZOATE | 6.3 | GMS |
| STEVIA POWDER | 5.3 | GMS |
| SUCRALOSE 100% POWDER | 1.6 | GMS |
| LEMON-LIME FLAVOR | 80.5 | GMS |
| CITRIC ACID | 66.5 | GMS |
| NIACIN | 0.79 | GMS |
| CHROMIUM POLYNICOTINATE | 0.03 | GMS |
| TOTAL: | 212.52 | GMS |
| WATER: | 4 | LT |

LT denotes Liters GMS denotes grams

Mix to dissolve, if any undissolved particulates are observed, filter through a coffee filter or equivalent mesh filter to remove visible material. For longer term storage, filter through a 0.2 micron or smaller filter prior to dilution or post dilution/bottling operations.

Add 9 parts of purified water to 1 part of the 10× concentrate, mix until homogeneous.

Fill into suitable containers for storage.

Although the invention has been described primarily in connection with special and preferred embodiments, it will be understood that it is capable of modification without departing from the scope of the invention. The following claims are intended to cover all variations, uses, or adaptations of the invention, following, in general, the principles thereof and including such departures from the present disclosure as come within known or customary practice in the field to which the invention pertains, or as are obvious to persons skilled in the field.

The invention claimed is:

1. An enhanced energy composition comprising per 500 mL:
a) caffeine ranging from 0.05 g to 0.25 g;
b) niacin ranging from 2 mg to 200 mg;
c) Vitamin C and/or Citric Acid, total amount ranging from 10 mg to 2000 mg;
d) Chromium Polynicotinate or Chromium Picolinate ranging from 0.005 mg to 2 mg; and
e) Electrolytes totaling from 10 mg to 5000 mg, including Potassium Sorbate and/or Sodium Benzoate as a preservative, with ranges as follows:
VI. Potassium Sorbate/Sodium Benzoate: 2 mg to 1000 mg
VII. Magnesium Chloride: 2 mg to 1000 mg
VIII. Sodium Chloride: 2 mg to 1000 mg
IX. Calcium Chloride: 2 mg to 1000 mg
X. Potassium Chloride: 2 mg to 1000 mg.

2. The composition of claim 1 wherein said caffeine is 125 mg.

3. The composition of claim 1 wherein said elemental chromium is 50 mcg.

4. The composition of claim 1 wherein said elemental calcium is 25.2 mg.

5. The composition of claim 1 wherein said total elemental potassium is 72.8 mg.

6. The composition of claim 1 wherein said elemental magnesium is 20.4 mg.

7. The composition of claim 1 wherein said total elemental sodium is 47.9 mg.

8. The composition of claim 1 wherein said citric acid is 831 mg.

9. The composition of claim 1 wherein said niacin is 9.9 mg.

10. The composition of claim 1 wherein said potassium sorbate is 179 mg.

11. The composition of claim 1 wherein said sodium benzoate is 78.9 mg.
